(19) United States
(12) Patent Application Publication
(10) Pub. No.: US 2014/0086793 A1
(43) Pub. Date: Mar. 27, 2014

(54) SYSTEMS AND METHODS FOR REDUCTION OF PATHOGENS IN A BIOLOGICAL FLUID USING VARIABLE FLUID FLOW AND ULTRAVIOLET LIGHT IRRADIATION

(71) Applicant: Hemalux Technologies LLC, Loveland, CO (US)

(72) Inventors: Ehud Ben-Hur, Loveland, CO (US); Heather Balji, Mahwah, NJ (US)

(73) Assignee: HEMALUX TECHNOLOGIES LLC, Loveland, CO (US)

(21) Appl. No.: 13/969,543

(22) Filed: Aug. 17, 2013

Related U.S. Application Data
(60) Provisional application No. 61/744,386, filed on Sep. 25, 2012.

(51) Int. Cl. A61L 2/00

(52) U.S. Cl. CPC A61L 2/0047
USPC 422/24; 250/438

(57) ABSTRACT

A system and method for reducing pathogens in a biological fluid such as whole blood or blood-derived products includes a pump configured to propagate the fluid through a serpentine-shaped flow path while exposing the fluid to UV irradiation. Extensive mixing of flow is accomplished by causing the pump to vary the flow of fluid. In embodiments, the pump may be operated to periodically switch the flow back and forth between a high flow rate and a slow flow rate. Alternatively, the pump may be periodically stopped or even cause the flow to reverse direction for short periods of time.

### CROSS-REFERENCE DATA

This patent application claims a priority date benefit from a related U.S. Provisional Patent Application No. 61/744,386 filed Sep. 25, 2012 by the same inventors and entitled “METHOD FOR PATHOGEN REDUCTION IN WHOLE BLOOD USING SHORT WAVELENGTH ULTRAVIOLET LIGHT”, which is incorporated herein in its entirety by reference.

### BACKGROUND OF THE INVENTION

The present invention relates to systems and methods for the UV-irradiation of a biological fluid for the purposes of reduction of pathogens therein. While the primary object of the invention is to treat blood, blood-based products and synthetic blood substitutes, the concepts of the present invention may be used for treating other fluids such as those encountered in beverage industries including dairy, distilling and brewing, as well as in water treatment industries including sewerage and purification systems.

The term “pathogens” is used broadly for the purposes of the present invention to include a variety of harmful microorganisms such as bacteria, viruses including among others a human immunodeficiency virus, a hepatitis A, B and C virus, parasites, molds, yeasts and other similar organisms which may be found in human or non-human blood and products derived from blood, as well as various other body fluids such (as for example milk) and synthetic fluids manufactured for use as replacements for any such body fluids or components thereof.

Blood transfusion in developed countries is very safe with regard to avoidance of transmitting of an infectious disease. This is primarily achieved by donor exclusion using questionnaires and screening for pathogens presence by means of serological methods and direct testing for nucleic acids. Despite these practices, there remains a risk of transmission of pathogens with the transfusion of cellular components of blood (such as red cells and platelets for example). This is at least in part because current screening tests leave a window of time after infection and before their sensitivity allows for detection of pathogens. In addition, screening does not takes place for rarely occurring pathogens or as yet unknown transmissible pathogens (Soland, E. M. et al. J. Am. Med. Assoc. 274: 1368-1373 (1995); Schreiber, G. B. et al. New Engl. J. Med. 334: 1685-1690 (1996); Valinsky, J. E. In: Blood Safety and Surveillance, Linden, J. V. and Bianco, C., Eds., Marcel Dekker, NY, 2001, pp. 185-219).

The use of pathogen reduction technologies has the potential of eliminating the remaining risks of transmission of infectious disease as a result of blood transfusion. Various approaches have been used to sterilize blood components (Ben-Hur, E. and B. Horowitz AIDS 10: 1183-1190 (1996); Ben-Hur, E. and R. P. Goodrich, In: Photodynamic Inactivation of Microbial Pathogens, Hamblin, M. R. and J. Gori, Eds. RSC Publishing, UK, 2011, pp. 233-263). The most promising methods are photochemical ones, two of which were approved by regulatory agencies for pathogen reduction in platelet concentrates. The Intercept method employs a psoralen and UVA light (Lin, L. et al. Transfusion 37: 423-435 (1997)) and the Mirasol method uses riboflavin and UVA+UVB light (Goodrich, R. P. et al. Transfusion Apheresis Sci. 35: 5-17 (2006)).

Short wavelengths ultraviolet light (UVC, 180-290 nm) is a known sterilizing agent that targets the nucleic acids of microorganisms (Setlow, R. B. and J. K. Setlow Proc. Natl. Acad. USA 48: 1250-1253 (1962)). It has been used for pathogen reduction in optically-transparent biological fluids such as plasma (Chin, S. et al. Blood 86: 4331-4336 (1995)) and is being studied also in platelet concentrates (Bashir, S. et al. Transfusion 53: 990-1000 (2013)). However, in opaque biological fluids such as red cell concentrates as well as in whole blood, UVC penetration is very limited due to absorption of UV irradiation by the red cells. As a result, all attempts to use UV irradiation for sterilizing whole blood or red cells have been unsuccessful so far.

Therefore, there is a need for an effective system and method for reducing pathogens in a biological fluid such as blood.

Attempts to irradiate blood or other opaque biological fluids with UV light have been described before. The exposure of a biological fluid to UV irradiation can result in damage to various components of the biological fluid, for example enzymes and other functional proteins. Therefore, the UV irradiation source should not be too powerful nor may the fluid be exposed to the UV radiation for too long, if one is to avoid damaging the components of the biological fluid.

To ensure that substantially all of the fluid receives a sufficient dose of UV radiation, it has been found that intensive mixing of the fluid to be treated during UV irradiation increases the efficiency of the irradiation process. A variety of devices that include static mixers placed in the fluid flow pathway have been proposed. The static mixers traditionally include elements protruding into the flow path such as alternating left- and right-handed helical elements that divert the flow to the left and then to the right while dividing it in half. Systems utilizing these static mixers typically include a constant flow pump (such as a peristaltic pump) operated to continuously propagate the biological fluid at a defined flow rate through an exposure chamber having a serpentine-shaped flow path. The flow path is equipped with internal static mixers designed to divide and rotate the flow of the biological fluid inside the flow path. Examples of such devices may be found in U.S. Pat. Nos. 1,683,877; 2,309,124; 3,527,940; 4,769,131; 4,898,702; 5,227,637; 5,433,738; 5,770,147; 6,113,566; 6,312,593; 6,464,936; 6,586,172; 6,951,548; 7,175,808; US Pat. Application Publications 2004/0039325; 2006/0270960; PCT publications WO1997046271; WO2000020045 and the GB patent No. 2200020—all incorporated herein by reference in their respective entireties.

While effective in mixing, such devices may cause excessive flow turbulence leading to hemolysis and other detrimental effects. They also introduce additional source of blood-contacting foreign surface which may activate certain elements in a biological fluid such as platelets to deposit over such foreign surfaces. The need therefore exists for systems and methods of reducing pathogens using UV light irradiation by a system with adjustable degree of mixing such that the intensity of mixing is sufficient for pathogen reduction but not excessive for causing damage to the biological fluid itself.

### SUMMARY OF THE INVENTION

Accordingly, it is an object of the present invention to overcome these and other drawbacks of the prior art by providing novel systems and methods for reduction of pathogens in a biological fluid by exposing the fluid to sufficient dose of UV irradiation.

It is another object of the present invention to provide systems and methods for effective mixing of a biological fluid while traveling through a flow path to expose the fluid to UV irradiation.

It is a further object of the present invention to provide novel systems and methods for reduction of pathogens in the biological fluid while minimizing the contact area of the foreign surface with the fluid itself.

It is yet a further object of the present invention to provide novel systems and methods for reducing pathogens in the biological fluid with the capability of adjusting the degree of mixing so as to optimally tune the mixing to the requirements of the UV exposure.

The system of the invention is designed for inactivation of pathogens in the biological fluid such as a unit of whole blood suitable for transfusion. The system includes a source of UV irradiation, such as in the UVC range—using for example short wavelengths of ultraviolet light. In embodiments, UVC light may be used, such as using a 254 nm wavelength. The method may include pumping a unit of whole blood shortly after its donation through a UVC-transparent flow path (a serpentine-shaped tube in some embodiments). The flow path may include one or more static mixer elements to cause intermittent or continuous mixing of the blood during its propagation through the flow path. The flow path may be exposed to UVC irradiation from a suitable source such as low pressure mercury lamps or a plurality of light emitting diodes—at an appropriate power density that allows sufficient UVC dose to be impinged on the blood such that sufficient inactivation of pathogens takes place. The process occurs within an exposure chamber and the rate of biological flow may be regulated by a suitable pump such as a peristaltic pump. The treated blood may then be collected in a new storage bag and the disposable flow path may be discarded after use.

To assure adequate mixing, the pump of the system may be operated to provide variable flow of the biological fluid through the flow path. To achieve this, an inherently variable flow pump may be used such as a diaphragm pump, or a constant flow pump (such as a peristaltic pump) may be operated in a variable flow manner. In embodiments, the pump may be operated to gradually change the flow on a periodic basis. In other embodiments, the pump may be operated to periodically provide a first flow rate for a predefined first period of time and then provide a second flow rate for a predefined second period of time. One of the first of second flow rate may be greater than, or less than the other flow rate. One of the flow rates may be zero when the pump is stopped altogether. The flow of the biological fluid may even reversed direction—but in that case the pump may be operated such that the cumulative flow resulting from the combination of the first flow rate and the second flow rate is still net positive so as to propagate the biological fluid from the inlet of the flow path towards the outlet thereof.

Variations of flow are designed to cause greater mixing of the biological fluid and provide for a uniform exposure of the biological fluid towards the UV irradiation source.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is block-diagram of the system of the present invention;

FIG. 2 is a side view of the flow path and the source of UV irradiation according to some embodiments of the invention;

FIG. 3 is a front view of the serpentine-shaped flow path;

FIG. 4 is a front view of a panel of UV light emitting diodes forming together a source of UV irradiation according to some embodiments of the present invention;

FIG. 5 shows a side view of a portion of the flow path of the system including static mixers for the flow of the biological fluid; and

FIGS. 6a and 6b show several ways to control the pump causing variable flow of the biological fluid through the flow path of the system.

### DETAILED DESCRIPTION OF THE PREFERRED EMBODIMENT OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

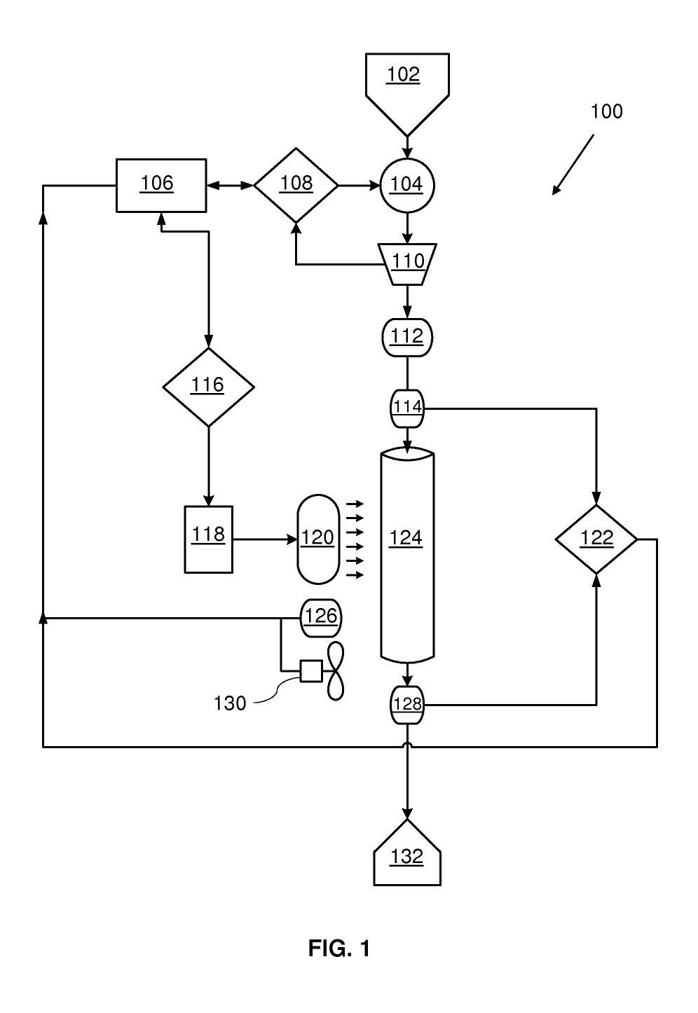

FIG. 1 shows a general block-diagram of the system 100 of the invention. A fluid supply source 102 may be used to draw the biological fluid from. Such fluid supply source may be a unit of blood collected from a donor for example. Biological fluid may be drawn from or gravity fed into a pump 104 suitable for the purposes of pumping the biological fluid. A variety of pumps may be used for the purposes of the present invention. In the case of processing blood or blood products, a biocompatible pump may be used such as a peristaltic pump, centrifugal pump, diaphragm pump or another blood-compatible pump. The pump 104 may be controlled by a pump controller 108, which in turn may be operable by a central control unit 106. The pump controller 108 may be operated to cause the pump 104 to propagate the biological fluid through the system with a variable rate as will be explained in more detail below.

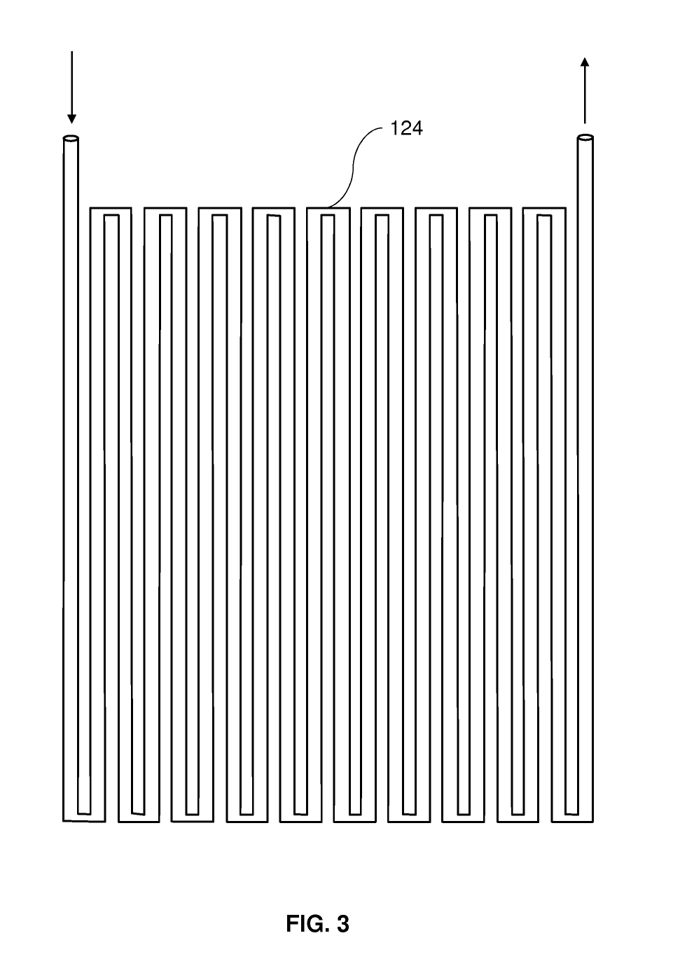

The biological fluid may be pumped by the pump 104 from the fluid supply 102 through the optional flow sensor 110 (operably connected to the pump controller 108), and further through an optional inlet pressure sensor 112 and an optional inlet temperature sensor 114 towards the fluid flow path 124, which is described in greater detail below with reference to FIG. 3.

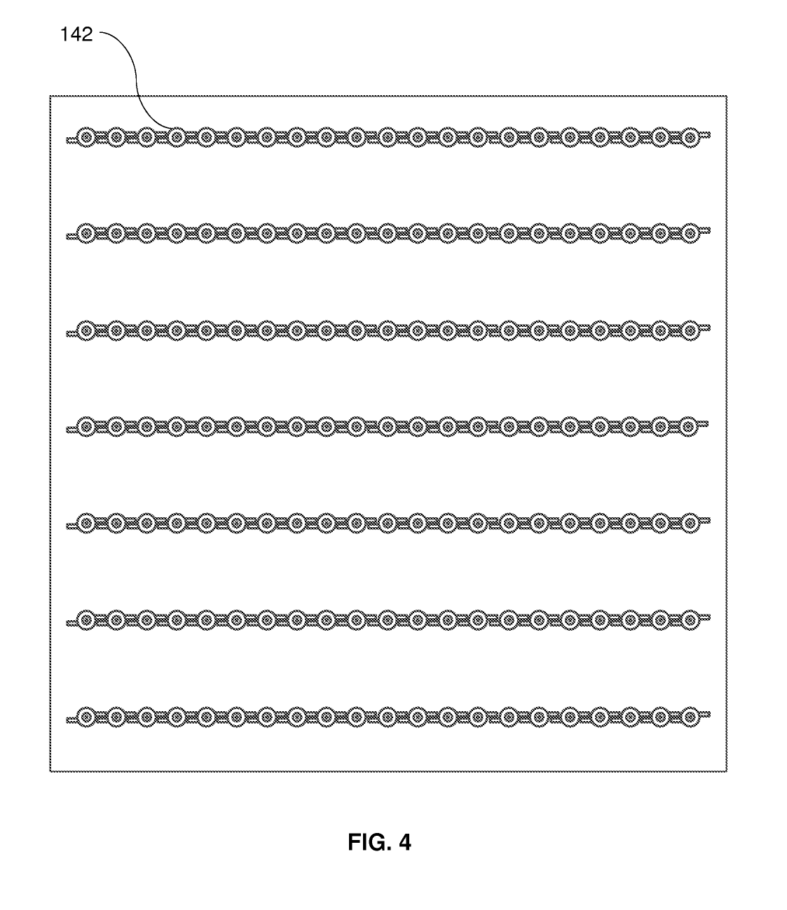

A UV source 120 may be used to provide UV irradiation suitable for reducing pathogens in the biological fluid. A plurality of UV irradiation lights or lamps may form together the source of UV irradiation 120. Utilizing such plurality as opposed to a single lamp allows a more uniform and spread-out UV exposure over a greater portion or preferably the entire flow path 124. The UV irradiation source 120 may be operated by a UV light source driver 118, which in turn is controlled by the light control circuit 116 operable by the central control unit 106. Further details of the UV irradiation source 102 are described below with reference to FIG. 4.

Following the exit from the flow path 124, the biological fluid is directed through an optional temperature sensor 128 towards the outlet fluid collection element 132, such as a blood collection bag for example.

Additional elements of the system 100 may include a fluid temperature control system 122, which may be operably connected to the inlet 114 and outlet 128 fluid temperature sensors to detect a potential increase in fluid temperature above a predetermined threshold. Such temperature increase may be caused by too much energy passed into the biological fluid from the source of UV irradiation 120. Undesirable increase in temperature may also be caused by improperly slow rate of flow through the flow path 124, which may cause an over-exposure of the biological fluid to UV irradiation. In any case, the optional fluid temperature control system 122 may be connected to the central control unit 106 and used to at least trigger an alarm. In some embodiments, the system 122 may be used to activate or regulate the active cooling of the flow path 124 and the biological fluid contained therein.

The cooling of the flow path 124 may be accomplished in a number of known ways. Passive cooling may be accomplished by using a heat sink or by providing passive vents to expose the outer surface of the flow path to atmosphere. Active cooling may be accomplished by providing one or more fans 130 or other cooling devices such as Peltier coolers, wherein such cooling devices may be activated upon the biological fluid reaching an upper limit of allowable safe temperature. When pumping blood or blood products, such upper temperature limit may be set at 38 degrees C.

In embodiments, the cooling elements of the system (such as cooling fans 130) may be activated for the entire duration of irradiating the biological fluid or for a portion thereof. The cooling elements may be activated on a predefined intermittent schedule or based on the feedback from the temperature sensors 114 and 128.

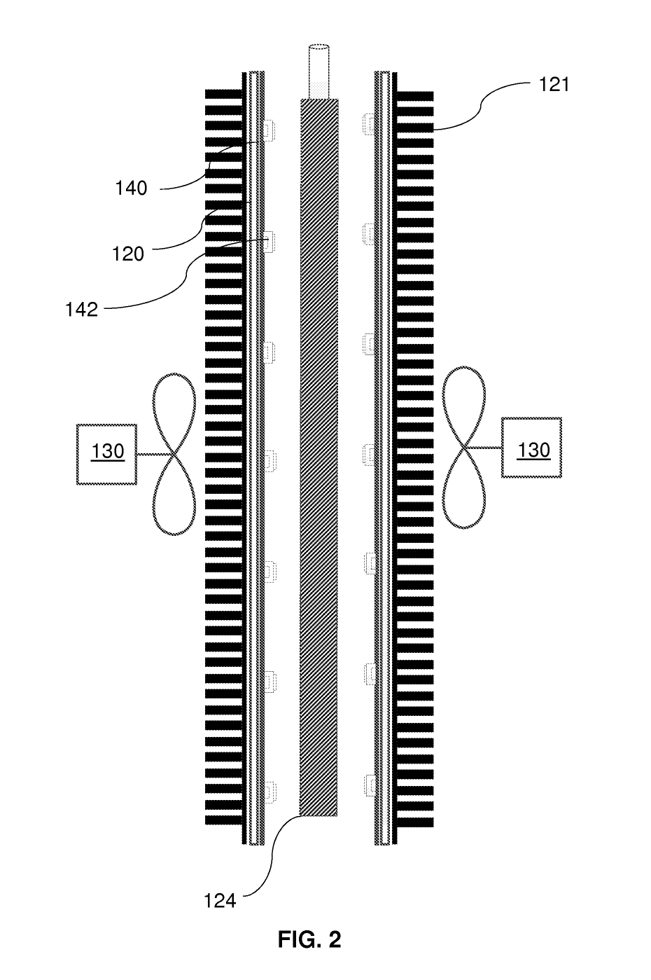

FIG. 2 shows a side view of the system 100 according to the block-diagram in FIG. 1. Shown here are the flow path 124 and two UV sources 120 on both sides thereof, each UV source comprising a printed circuit board 140 with rows of surface mounted UV light emitting diodes (LED) 142 on one side and PCB heat sink 121 on the other side of the circuit board. Cooling fans 130 on one or both sides of the flow path 124 may be used to cool one or both the flow path 124 itself as well as the LEDs 142 to dissipate heat generated by the LEDs 142 during the time of their activation.

The details of the flow path 124 are better seen on the front view thereof shown in FIG. 3. The flow path may be selected to be sufficiently long to provide for sufficient UV exposure for the biological fluid propagating therethrough. The length of the flow path may vary from about 1 meter to about 20 meters. In embodiments, the length of the flow path may depend on the diameter thereof, the rate of biological fluid flow, the strength of UV irradiation, desired efficacy of pathogen reduction (“log kill” limit) and other factors. In one embodiment of the invention, the length of the flow path 124 may be selected to be from about 4 meters to about 20 meters. The term “about” is used herein and throughout the specification to mean a deviation of +/− 30% of the cited parameter. In embodiments, the length of the flow path may be selected to be about 4 meters, about 6 meters, about 8 meters, about 10 meters, about 12 meters, about 14 meters, about 16 meters, about 18 meters, about 20 meters or any length in-between these numbers.

The cross-sectional shape of the flow path may be selected to be flat, oval, or round. In case of a round cross-sectional shape, the internal diameter of the flow path 124 may be selected to be constant or variable along its length. In embodiments, a constant internal diameter may be selected to be from about 1 mm to about 8 mm. In embodiments, the internal diameter of the flow path 124 may be selected to be about 1 mm, about 2 mm, about 3 mm, about 4 mm, about 5 mm, about 6 mm, about 7 mm, about 8 mm, or any diameter in-between these numbers.

To accomplish substantial length of the flow path, various methods of folding the flow path into a compact structure may be used. One such method is to form the flow path in a serpentine shape as shown in FIG. 3 with a well-defined inlet and outlet indicated by arrows in FIG. 3. The serpentine-shaped flow path 124 may be formed along a single flat plane so as to allow its exposure on both sides to the sources of UV irradiation 120 as shown in FIG. 2. Alternatively, the serpentine shape may be wrapped about a centrally-placed source of UV irradiation or otherwise presented in a compact way to make the entire system easy to use.

In embodiments, all or some elements of the system which are in direct contact with the biological fluid may be made disposable or reusable. In some embodiments, the flow path 124 may be made disposable. In other embodiments, the flow path 124 along with the tubing for the peristaltic pump 104 as well as the necessary sensors may all be formed as a disposable cassette or a cartridge for easy handling before, during and after use. Such disposable cassette may have provisions to “plug into” the rest of the system including reusable UV irradiation source, the active portion (rollers) of the pump 104, and all the elements of the above described control system. Once assembled, the disposable portion of the system may be attached at the inlet to the source 102 of biological fluid and at the outlet to the fluid collection element 132.

The serpentine-shaped flow path 124 may be assembled from individual straight sections or made as a single unit by any suitable technique such as thermoforming, molding etc. In embodiments, the fluid flow path may be made from a UV-transparent material such as glass, and in particular quartz glass. Alternatively, the flow path may be formed from plastics such as organic polymers, co-polymers and the like such as but not limited to cellulose products, PTFE, FEP, PVC and PE. In general, these materials have UV transmission properties in the range from 30 to 95% for a typical wall thickness, which may generally be between about 0.3 mm and about 2 mm.

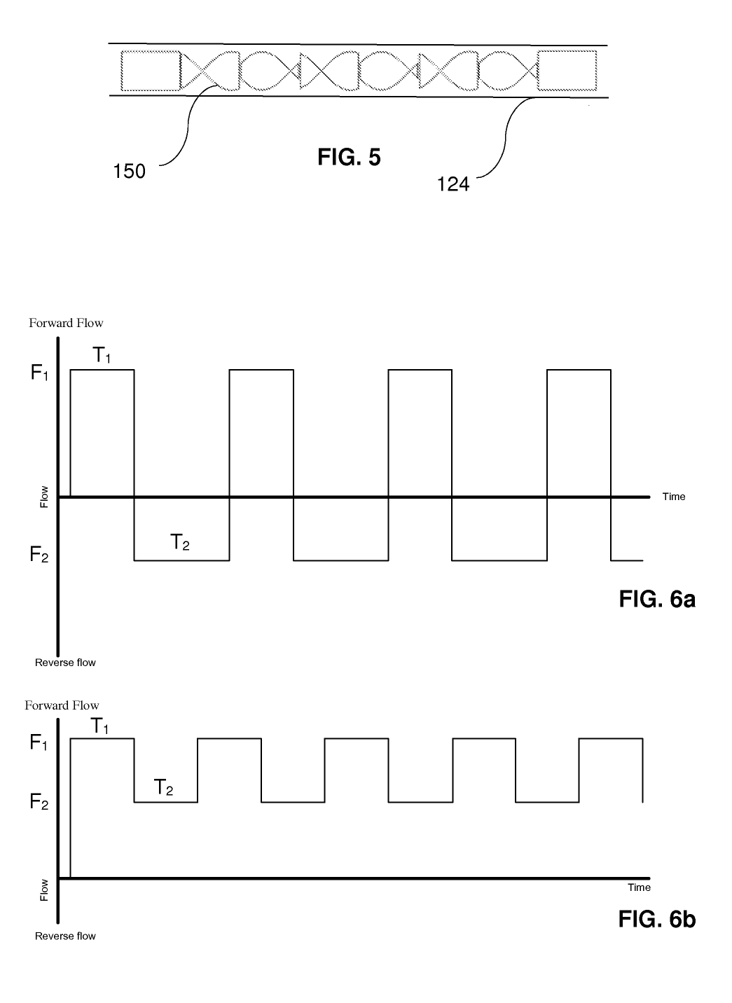

The flow path 124 may have an open round internal cross-section in some embodiments while in other embodiments the flow path 124 may contain one or more static mixers 150 as seen in FIG. 5. These static mixers 150 may be made according to the descriptions in the cited above patents. Generally, the static mixers 150 may be designed as left- or right-turn helical elements positioned inside the flow path 124 and configured to divide the flow of the biological fluid in two flows while simultaneously imparting a flow turn along their helical spiral shape. Alternating right- and left-turned static mixers may accomplish the goal of effective mixing of the biological fluid propagating through the flow path 124.

In embodiments, the static mixers 150 may be firmly positioned inside the flow path 124. In other embodiments, the static mixers 150 may be rotatably supported in the flow path 124. In this case, varying the flow by the pump 104 during operation of the system may cause the static mixers 150 to turn inside the flow path—causing further mixing of the biological fluid.

In further embodiments of the invention, the static mixers 150 may be made using a rigid biocompatible and UV-resistant material, such as the material used for forming the flow path 124 itself as described above. In other embodiments, the material of the static mixers 150 may be made UV-transparent to further enhance irradiation of the biological fluid and prevent any shadows from the static mixers 150 from limiting such exposure.

In other embodiments, the material for the static mixers 150 may be selected to be flexible and partially elastic. In this case, periodic varying of the flow by the pump 104 may cause the blades of the mixers 150 to periodically deflect back and forth during operation of the system, whereby further improving the mixing of the biological fluid inside the flow path 124.

FIG. 4 shows a front view of the source of UV irradiation suitable at least in some embodiments of the invention. Shown here is a panel such as a printed circuit board, which contains a plurality of UV irradiation elements such as low pressure mercury lamps or in some embodiments, UV light emitting diodes 142. Such elements may be arranged in rows and positioned next to the serpentine-shaped flow path 124 as described above.

Surface-mounted LEDs may be advantageously used for the purposes of this invention due to their small size and effective UV irradiation capacity. UV emitting LEDs may be closely positioned next to each other and together provide a source of UV irradiation which is uniformly spread out along at least a part of the flow path 124. In embodiments, UV irradiation in the C range of wavelengths may be used. In particular, UV LEDs irradiating The UV light in the range of wavelengths from about 240 nm to about 280 nm with a peak wavelength at about 254 nm to about 265 nm may be used. The light output of such LEDs may be about 20 mW, and a viewing angle is about 120°. High efficiency LEDs manufactured by Crystal IS (Green Island, N.Y.) may be used for the purposes of the present invention. These LEDs emit over 90% of their energy at the desired peak wavelength.

The peak wavelength selection is dictated by the need to be at or close to the absorption peak of nucleic acid which is 265 nm, which is critical for inactivation of various pathogens and microorganisms.

In embodiments, two panels of LEDs may be positioned on both sides of the flow path 124. To further increase the efficacy of UV irradiation, the inner surface of the LED panels may be covered with a reflective material so as to redirect stray UV irradiation back towards the flow path 124.

The rate of biological fluid flow may be regulated by the pump 104 placed at the inlet of the flow path 124. As the UV irradiation of the flow path 124 may be constant, the UV light dose absorbed by the biological fluid may be regulated by the flow rate thereof through the flow path 124 and by the number of times the biological fluid is caused to propagate through the flow path 124. Multiple circulations of the biological fluid through the flow path 124 may be accomplished in some embodiments by using a 3-way valve at the exit of the flow path and redirecting the biological fluid back towards the inlet of the flow path (not shown in the drawings). The overall rate of biological fluid propagation through the flow path 124 may be selected such that the UV light dose absorbed by the fluid may be sufficient to reduce the level of pathogens or infectious agent contained therein. Maximum inactivation of pathogens is preferred but only up to a safe level above which damage to the biological fluid may occur. For example, to minimize damage to the red cells, platelets and plasma proteins, a dose of 0.1 J/cm² of UVC light may be used.

In use, the system of the invention may be operated in the following way. Initial supply of the biological fluid may be positioned in the fluid supply source 102. The entire fluid contacting circuit as described above may be primed or filled with saline or the biological fluid itself may be pumped therethrough by the pump 104. The process may then be initiated by activating the source of UV irradiation 120 and the pump 104.

Pumping of the biological fluid through a serpentine-shaped flow path 124 causes it to be exposed to UV irradiation. Active mixing of the fluid is designed to bring pathogens to the surface of the flow and inactivate them by UV light. Flow mixing may be achieved by varying the flow through the pump 104 in a number of advantageous ways depending on the nature of the pump.

Inherently pulsatile flow may be produced by the pump if it is made as a diaphragm or piston pump. The diaphragm may be activated in a reciprocal motion by a movement of the driving piston or by supplying gas or fluid pressure alternating with vacuum on the opposite side of the diaphragm. The rate of diaphragm or piston movement may be controlled by the pump controller 108 in order to cause sufficient but not excessive mixing of the biological fluid in the flow path 124.

A constant flow biocompatible pump design may also be used for the system of the present invention. Such pump may be a centrifugal pump or a peristaltic pump (also referred to as a roller pump). In this case, the rotational speed of the pump may be controlled by the pump controller 108 in order to cause variable flow of the biological fluid through the pump 104 and through the flow path 124.

The flow of the biological fluid may be varied on an intermittent or a periodic and repeatable basis. The fluid flow may also be varied from time to time—alternating with periods of constant flow. In embodiments, the fluid flow may follow a predefined program, such as oscillating based on a sinusoidal waveform.

In other embodiments, the flow may include a first flow rate maintained for a first period of time following by a second flow rate maintained for a predefined second period of time. This arrangement is generally shown in FIGS. 6a and 6b.

FIG. 6a shows a method of varying the flow of the biological fluid through the flow path 124 which includes a constant first forward flow F₁ maintained for a first period of time T₁. The term “forward” is used to describe the fluid propagating from the inlet of the flow path 124 towards its outlet. This flow rate may be maintained by a peristaltic pump by maintaining the constant speed of roller rotation at a first speed level. The pump operation may then be switched to a second flow rate F₂ and maintained for a second period of time T₂. In this example, the second flow rate is causing the biological fluid to reverse direction to flow from the outlet of the flow path 124 towards its inlet. Sudden change in flow direction is designed to provide for effective mixing inside the flow path 124.

Importantly, both the first and the second flow rates and both the first and the second durations may be selected to assure that the cumulative flow is still net positive—defined as to propagate the biological fluid in the forward direction. This may be accomplished by having an area under the curve (AUC) above the zero line to be greater than the AUC below the zero line. For example, this may be accomplished by selecting the first (forward) flow rate to be greater than the second (reverse) flow rate and/or by selecting the first period of time to be longer than the second period of time. After the second period of time has elapsed, the pump controller 108 may be configured to repeat the sequence of two flow rates again—immediately (shown in FIG. 6a) or after a predetermined delay (not shown).

The pump controller 108 may also be configured to reverse the direction of flow in a gradual way—by slowing down and then gradually reversing the rotation of the rollers in a peristaltic pump. This may be needed to avoid sudden spikes in flow turbulence, which may be caused by abrupt stopping of the pump and reversing its direction.

Less aggressive but still effective mixing of biological fluid may be accomplished by selecting the second flow rate to be zero (pump is stopped) or also propagating the fluid in a forward direction. In this case, fast (F₁) and slow (F₂) flow rates may be used—as seen in FIG. 6b. A gradual transition between the first and the second flow rates may be deployed to reduce shear stress on the biological fluid.

There is also provided a method for operating a pump, which is configured to propagate a biological fluid through a UV-transparent flow path. The flow path is in turn adapted for exposure of the biological fluid to UV light irradiation in order to cause reduction of pathogens therein. The method of the invention comprises a step of operating the pump in such a manner so as to cause mixing of the biological fluid and improving exposure thereof to said UV light irradiation. The manner of operating the pump includes varying a flow of the biological fluid through the pump while propagating the fluid through the flow path.

### EXAMPLE

An experiment was conducted to demonstrate the ability of a device constructed as described above to inactivate a model virus in blood. In this experiment, whole blood was spiked with bacteriophage φ6, which is similar in structure to HIV (the genome is RNA of about 7000 nucleotides and is lipid-enveloped). The final titer of virus in the blood was about 10 logs. The blood was pumped through the device at various flow rates and samples were withdrawn at each flow rate. The blood samples were diluted 10-fold with saline and centrifuged at 3,000 rpm. The supernatant was then assayed for virus titer after 10-fold serial dilutions, on a lawn of the host bacterium (Pseudomonas syringae), by scoring the number of plaques formed on the Petri dishes. The extent of virus inactivation (log₁₀) was calculated by comparing virus titer in treated blood with that of the control, untreated blood.

UVC power density during operation of the device was 2.2 mW/cm². The UVC light dose to which the blood was exposed was inversely related to the rate of blood flow, which varied from on average of about 5 ml/min to about 20 ml/min. The volume of the blood exposed to UVC inside the device was 100 ml. The transit time of the blood was therefore 20 min at 5 ml/min and 5 min at 20 ml/min. The total UVC dose calculated from the power density and transit time varied from 0.66 to 2.64 J/cm². The results are shown below:

| UVC light dose (J/cm²) | Virus inactivation (log₁₀) |
|---|---|
| 0.66 | 1.2 |
| 0.99 | 1.5 |
| 1.32 | 2.0 |
| 2.64 | 3.2 |

These results demonstrate the ability of the device to inactivate over 99.9% of a model RNA virus in a UVC light dose-dependent manner.

The herein described subject matter sometimes illustrates different components or elements contained within, or connected with, different other components or elements. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for reduction of pathogens in a biological fluid, the system comprising:
a source of UV light irradiation,
a UV-transparent flow path for the biological fluid, said flow path having an inlet and an outlet, said flow path is configured to expose the biological fluid propagating therethrough to UV light from said source of UV light irradiation upon activation thereof,
a pump operably connected to said flow path, said pump is configured to cause the biological fluid to propagate from said inlet towards said outlet, and
a pump control system configured to operate said pump in a variable flow mode including reversal of flow direction to propagate from said outlet towards said inlet so as to cause mixing of the biological fluid while in said flow path.

2. The system as in claim 1, wherein said variable flow mode is configured for a periodic and repeatable change of flow rate of the biological fluid through said flow path.

3. The system as in claim 2, wherein said pump control system is configured to operate said pump in a repeatable sequence of a first flow rate followed by a second flow rate of the biological fluid through said flow path.

4. The system as in claim 3, wherein said first rate of flow and said second rate of flow causing the biological fluid to propagate from said inlet to said outlet of said flow path, one of said first rate of flow or said second rate of flow is greater than another, whereby switching between said flow rates causes greater mixing of the biological fluid while inside said flow path.

5. The system as in claim 3, wherein said switching between said flow rates is done gradually so as to avoid excessive turbulence of said biological fluid.

6. (canceled)

7. The system as in claim 3, wherein said pump control system is further configured to cause the total flow resulting cumulatively from said first rate of flow and said second rate of flow to be net positive in propagating the biological fluid from said inlet to said outlet of said flow path.

8. The system as in claim 1, wherein said flow path is generally round with an inside diameter ranging from about 1 mm to about 8 mm.

9. The system as in claim 8, wherein said inside diameter of said flow path is about 3 mm.

10. The system as in claim 8, wherein said flow path has a length ranging from about 4 meters to about 12 meters.

11. The system as in claim 10, wherein said length of the flow path is about 8 meters.

12. The system as in claim 1 further comprising at least one static mixer placed inside said flow path to cause additional mixing of the biological fluid and improved exposure to said source of UV light irradiation.

13. (canceled)

14. A method for reducing pathogens in a biological fluid, said method comprising a step of propagating the biological fluid through a UV-transparent flow path to expose thereof to UV light irradiation, wherein said propagating is conducted by at least once varying a rate of flow of the biological fluid through said flow path to cause mixing of the biological fluid and improved UV irradiation exposure.

15. The method as in claim 14, wherein said step of propagating the biological fluid including periodic changing of flow rate of the biologic fluid through said flow path.

16. The method as in claim 14, wherein said step of propagating the biological fluid including intermittent stopping or reversing of flow direction of the biological fluid in said flow path.

17. A method for operating a pump, said pump is configured to propagate a biological fluid through a UV-transparent flow path adapted for exposure of the biological fluid to UV light irradiation to cause reduction of pathogens therein, said method comprising a step of operating said pump to cause mixing of the biological fluid and improving exposure thereof to said UV light irradiation by varying a flow of the biological fluid through the pump while propagating through said flow path.
